(19) United States
(12) Patent Application Publication
(10) Pub. No.: US 2025/0062025 A1
(43) Pub. Date: Feb. 20, 2025

(54) HYBRID MACHINE LEARNING MODELS FOR IMPROVED DIAGNOSTIC ANALYSIS

(71) Applicant: IDEXX Laboratories, Inc., Westbrook, ME (US)

(72) Inventors: Jeremy HAMMOND, Standish, ME (US); Mathew Furtney, Freeport, ME (US); Carl Ericson, Westbrook, ME (US); David Steines, Scarborough, ME (US); Scott Bender, Cape Elizabeth, ME (US)

(21) Appl. No.: 18/807,045

(22) Filed: Aug. 16, 2024

Related U.S. Application Data
(60) Provisional application No. 63/533,407, filed on Aug. 18, 2023.

(51) Int. Cl. G16H 50/20; G06N 20/00

(52) U.S. Cl. CPC G16H 50/20; G06N 20/00

(57) ABSTRACT

Methods and systems for performing diagnosis using patient sample data are described. The method comprises receiving patient sample data at a point of care, analyzing the received patient sample data at the point of care, and evaluating a first result of analyzing the received patient sample data at the point of care to determine whether a criterion for advanced analysis at a central reference laboratory is met. In a case where the criterion for advanced analysis is met, the method further comprises reflexing the received patient sample data to the central reference laboratory for the advanced analysis, receiving, at the point of care, a second result of the advanced analysis from the central reference laboratory, and displaying at least one of the first result or the second result on a display at the point of care.

### CROSS REFERENCE TO RELATED APPLICATIONS

This application claims priority to U.S. Provisional Patent Application No. 63/533,407 filed Aug. 18, 2023, the entire disclosure of which is hereby incorporated herein by reference.

### TECHNICAL FIELD

The present disclosure generally relates to diagnostic models for human and veterinary applications, and more specifically relates to hybrid machine learning models that are maintained at a point-of-care system and a centralized system simultaneously.

### BACKGROUND

Diagnostic instruments have been used for decades in both human and veterinary applications. These instruments include hematology analyzers, chemistry analyzers, and other instruments that determine certain physiological properties of patients. These diagnostic instruments may be located at a point-of-care facility, such as a clinic or an on-site laboratory, or at a centralized location.

### SUMMARY

A diagnostic system providing medical insights at the point-of-care, defined here as a point-of-care (POC) system, generally incorporates on-board algorithms that use machine learning models to provide diagnostic results to clinicians (veterinarians, doctors, laboratory technicians etc.) using hematology, clinical chemistry, immunoassay, and urinalysis, for example. Alternatively, the clinician can collect samples from a patient and send them to a central reference laboratory (CRL) for diagnostic analysis, potentially including manual evaluation of data obtained from the samples. In some cases, the POC system may include a diagnostic analyzer where some of the data (patient sample) is collected and analyzed locally; and the same data is sent to a CRL for professional clinical analysis. Such cases can include X-ray digital radiography, electrocardiograms (ECGs), and digital blood morphology, for example.

POC systems provide real-time analysis and feedback, but data from the POC systems are not often reviewed for expert interpretation by a clinical pathologist or radiologist. CRL-based diagnostic systems and processes can provide analysis by board certified professionals, but generally require significantly more time than POC systems. Further, while POC systems can have some simple machine learning models to improve diagnostic performance, they have limited computing power and cannot run more complex and advanced machine learning models, or continue to refine the learned models through continuous learning. Moreover, the simple machine learning models available in the POC systems cannot match the performance of the more complex and advanced machine learning models that are available at the CRL.

Thus, there exists a need for a new approach that integrates POC systems and CRL-based diagnostic systems and processes to provide the advantages of both POC and CRL systems (quick and simple diagnosis at the POC with case-by-case based advanced diagnosis at the CRL) and permits continuous refinement of the machine learning models present in the POC and CRL systems.

At least the above-discussed needs are addressed, and technical solutions are achieved in the art, by various aspects of the present disclosure.

Some aspects of the present disclosure pertain to a method executed by a programmed data processing device system comprising: receiving patient sample data at a point of care; analyzing the received patient sample data at the point of care; evaluating a first result of analyzing the received patient sample data at the point of care to determine whether a criterion for advanced analysis at a central reference laboratory is met; and, in a case where the criterion for advanced analysis is met, reflexing the received patient sample data to the central reference laboratory for the advanced analysis, receiving, at the point of care, a second result of the advanced analysis from the central reference laboratory, and displaying at least one of the first result or the second result on a display at the point of care.

In some aspects, the method further includes, in a case where the criterion for advanced analysis is not met, displaying the first result on the display at the point of care.

In some aspects, the method further includes analyzing the received patient sample data using a machine learning model at the point of care system. In some aspects, the machine learning model is a convolution neural network.

In some aspects, the method further includes analyzing the reflexed patient sample data using a machine learning model at the central reference laboratory. In some aspects, the machine learning model is a convolution neural network.

In some aspects, the method further includes: receiving a plurality of patient sample data at the central reference laboratory; training a first machine learning model using the plurality of patient sample data; and deploying the trained first machine learning model in the point of care system to analyze the patient sample data received at the point of care.

In some aspects, the method further includes: receiving a plurality of patient sample data at the central reference laboratory; training a second machine learning model using the plurality of patient sample data; and analyzing the reflexed patient sample data at the central reference laboratory using the trained second machine learning model.

In some aspects, the criterion includes one or more of (i) a case where the first result indicates that the received patient sample data is abnormal, (ii) a case where a difference between the first result and a reference value is greater than a first threshold, (iii) a case where a confidence level associated with the first result is less than a second threshold, (iv) a case where the first result cannot be obtained by analysis at the point of care, or (v) a case where the first result indicates a diagnostic condition that requires advanced analysis at the central reference laboratory.

Some aspects of the present disclosure pertain to a diagnostic system comprising: a memory configured to store instructions; and a processor communicatively connected to the memory and configured to execute the stored instructions to: receive patient sample data at a point of care; analyze the received patient sample data at the point of care; evaluate a first result of analyzing the received patient sample data at the point of care to determine whether a criterion for advanced analysis at a central reference laboratory is met; and in a case where the criterion for advanced analysis is met: reflex the received patient sample data to the central reference laboratory for the advanced analysis, receive, at the point of care, a second result of the advanced analysis from the central reference laboratory, and display at least one of the first result or the second result on a display at the point of care.

In some aspects, the processor is further configured to execute the stored instructions to, in a case where the criterion for advanced analysis is not met, display the first result on the display at the point of care.

In some aspects, the processor is further configured to execute the stored instructions to analyze the received patient sample data using a machine learning model at the point of care system. In some aspects, the machine learning model is a convolution neural network.

Some aspects of the present disclosure pertain to a hybrid diagnostic system comprising: a first memory configured to store first instructions; a first processor communicatively connected to the first memory and configured to execute the stored first instructions at a point of care to: receive patient sample data at the point of care; analyze the received patient sample data at the point of care; evaluate a first result of analyzing the received patient sample data at the point of care to determine whether a criterion for advanced analysis at a central reference laboratory is met; and, in a case where the criterion for advanced analysis is met: reflex the received patient sample data to the central reference laboratory for the advanced analysis, receive, at the point of care, a second result of the advanced analysis from the central reference laboratory, and display at least one of the first result or the second result on a display at the point of care; a second memory configured to store second instructions; and a second processor communicatively connected to the second memory and configured to execute the stored second instructions at the central reference laboratory to: receive the reflexed patient sample data from the point of care; analyze the received patient sample data at the central reference laboratory to generate the second result; and transmit, to the point of care, the second result of the advanced analysis from the central reference laboratory.

In some aspects, the first processor is further configured to execute the stored first instructions to, in a case where the criterion for advanced analysis is not met, display the first result on the display at the point of care.

In some aspects, the first processor is further configured to execute the stored first instructions to analyze the received patient sample data using a machine learning model at the point of care system. In some aspects, the machine learning model is a convolution neural network.

In some aspects, the second processor is further configured to execute the stored second instructions to analyze the reflexed patient sample data using a machine learning model at the central reference laboratory. In some aspects, the machine learning model is a convolution neural network.

In some aspects, the second processor is further configured to execute the stored second instructions to: receive a plurality of patient sample data at the central reference laboratory; train a first machine learning model using the plurality of patient sample data; and deploy the trained first machine learning model in the point of care system to analyze the patient sample data received at the point of care.

In some aspects, the second processor is further configured to execute the stored second instructions to: receive a plurality of patient sample data at the central reference laboratory; train a second machine learning model using the plurality of patient samples; and analyze the reflexed patient sample data at the central reference laboratory using the trained second machine learning model.

Some aspects of the present disclosure pertain to a non-transitory computer readable storage medium configured to store a program that executes a diagnostic method, the method comprising: receiving patient sample data at a point of care; analyzing the received patient sample data at the point of care; evaluating a first result of analyzing the received patient sample data at the point of care to determine whether a criterion for advanced analysis at a central reference laboratory is met; and, in a case where the criterion for advanced analysis is met: reflexing the received patient sample data to the central reference laboratory for the advanced analysis, receiving, at the point of care, a second result of the advanced analysis from the central reference laboratory, and displaying at least one of the first result or the second result on a display at the point of care.

Some aspects of the present disclosure pertain to another method executed by a programmed data processing device system, the another method comprising: receiving patient sample data at a point of care; analyzing the received patient sample data at the point of care using a machine learning model; determining whether a criterion for using the received patient sample data for updating the machine learning model is met based on a result of analyzing the received patient sample data at the point of care; and, in a case where the criterion for updating the machined learning model is met: transmitting the received patient sample data to a central reference laboratory, updating, at the central reference laboratory, the machine learning model by using the patient sample data as new training data for the machine learning mode, and deploying, at the point of care, the updated machine learning model from the central reference laboratory.

In some aspects of the another method, the criterion includes one or more of (i) a case where the result indicates that the received patient sample data is relevant to a diagnostic prediction output by the machine learning model, (ii) a case where the result indicates that the received patient sample data is relevant to a diagnostic prediction having sparse training data, or (iii) a case where a confidence level associated with the result is less than a threshold.

In some aspects, the another method further includes: defining a sampling strategy for transmitting new training data to the central reference laboratory for updating the machine learning model; and transmitting the received patient sample data to the central reference laboratory in accordance with the sampling strategy.

In some aspects of the another method, the sampling strategy includes at least one of random sampling, stratified sampling, cluster sampling, importance sampling, uncertainty sampling, and/or active learning.

Subsets or combinations of various aspects of the disclosure described above provide further aspects of the disclosure.

### BRIEF DESCRIPTION OF THE DRAWINGS

It is to be understood that the attached drawings are for purposes of illustrating aspects of various aspects of the disclosure and may include elements that are not to scale. It is noted that like reference characters in different figures refer to the same objects.

FIG. 1 shows a computing device system, according to some aspects of the disclosure.

FIG. 2 shows another computing device system, according to some aspects of the disclosure.

FIG. 3 shows hardware architecture of a hybrid diagnostic system, according to some aspects of the disclosure;

FIG. 4 shows a flowchart of a method of performing diagnostic analysis using the hybrid diagnostic system, according to some aspects of the disclosure;

FIG. 5 shows a block diagram of a clinical diagnostic software system, according to some aspects of the disclosure; and

FIG. 6 shows a flowchart of a method of updating the machine learning models, according to some aspects of the disclosure.

### DETAILED DESCRIPTION

In some aspects of the disclosure, the computer systems described herein execute methods for implementing hybrid machine learning models that perform simpler diagnostics at the POC and automatically reflex the patient sample data to the CRL for advanced diagnostics when appropriate. The computer systems described herein also permit the machine learning models to be refined over time, using new patient sample data, to ensure that the models reflect the changes occurring in the real world. It should be noted that the aspects or embodiments of the present disclosure are not limited to these or any other examples provided herein, which are referred to for purposes of illustration only.

In this regard, in the descriptions herein, certain specific details are set forth in order to provide a thorough understanding of various aspects of the disclosure. However, one skilled in the art will understand that the invention may be practiced at a more general level without one or more of these details. In other instances, well-known structures have not been shown or described in detail to avoid unnecessarily obscuring descriptions of various aspects of the disclosure.

Any reference throughout this specification to one “aspect” or “embodiment”, an “aspect” or “embodiment,” an example “aspect” or “embodiment,”, an illustrated “aspect” or “embodiment,” a particular “aspect” or “embodiment,” and the like means that a particular feature, structure, or characteristic described in connection with the aspect or embodiment is included in at least one aspect or embodiment. Thus, any appearance of the phrase in one “aspect” or “embodiment,” in an “aspect” or “embodiment,” in an example “aspect” or “embodiment,” in this illustrated “aspect” or “embodiment,” in this particular “aspect” or “embodiment,” or the like in this specification is not necessarily all referring to one aspect or embodiment or a same aspect or embodiment. Furthermore, the particular features, structures or characteristics of different aspects or embodiments of the disclosure may be combined in any suitable manner to form one or more other aspects or embodiments of the disclosure. Further, the term aspect or embodiment may be used interchangeably.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, some aspects of the disclosure may be implemented at least in part by a data processing device system configured by a software program. Such a program may equivalently be implemented as multiple programs, and some or all of such software program(s) may be equivalently constructed in hardware.

Further, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device,” the word “machine,” the word “system,” and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various aspects of the disclosure that a device or machine or device system resides entirely within a same housing to exclude aspects of the disclosure where the respective device, machine, system, or device system resides across different housings. The word “device” may equivalently be referred to as a “device system” in some aspects of the disclosure.

The phrase “derivative thereof” and the like is or may be used herein at times in the context of a derivative of data or information merely to emphasize the possibility that such data or information may be modified or subject to one or more operations. For example, if a device generates first data for display, the process of converting the generated first data into a format capable of being displayed may alter the first data. This altered form of the first data may be considered a derivative of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended to include these and like changes, regardless of whether or not the phrase “derivative thereof” or the like is used in reference to the information or data, unless otherwise required by context. As indicated above, usage of the phrase “or a derivative thereof” or the like merely emphasizes the possibility of such changes. Accordingly, the addition of or deletion of the phrase “or a derivative thereof” or the like should have no impact on the interpretation of the respective data or information. For example, the above-discussed color-coded bar chart may be considered a derivative of the respective first data or may be considered the respective first data itself.

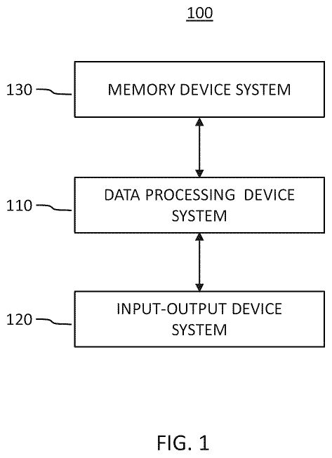

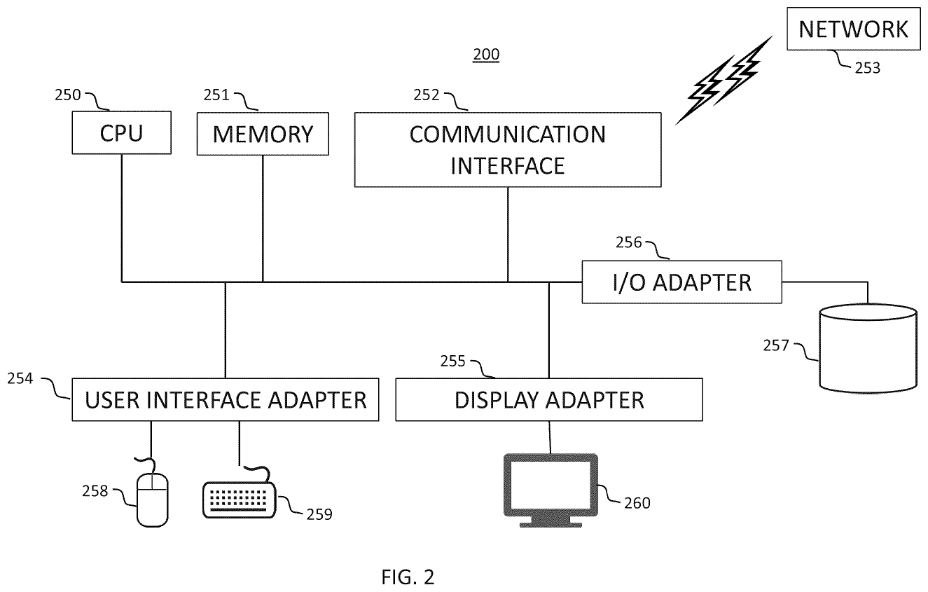

The term “program” in this disclosure should be interpreted to include one or more programs including a set of instructions or modules that may be executed by one or more components in a system, such as a controller system or data processing device system, to cause the system to perform one or more operations. The set of instructions or modules may be stored by any kind of memory device, such as those described subsequently with respect to the memory device system 130, 251, or both, shown in FIGS. 1 and 2, respectively. In addition, this disclosure may describe or similarly describe that the instructions or modules of a program are configured to cause the performance of an action. The phrase “configured to” in this context is intended to include at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data includes modifications to that information or data. For example, “data X” may be encrypted for transmission, and a reference to “data X” is intended to include both its encrypted and unencrypted forms, unless otherwise required or indicated by context. However, non-usage of the phrase “or a derivative thereof” or the like nonetheless includes derivatives or modifications of information or data just as usage of such a phrase does, as such a phrase, when used, is merely used for emphasis.

Further, the phrase “graphical representation” used herein is intended to include a visual representation presented via a display device system and may include computer-generated text, graphics, animations, or one or more combinations thereof, which may include one or more visual representations originally generated, at least in part, by an image-capture device.

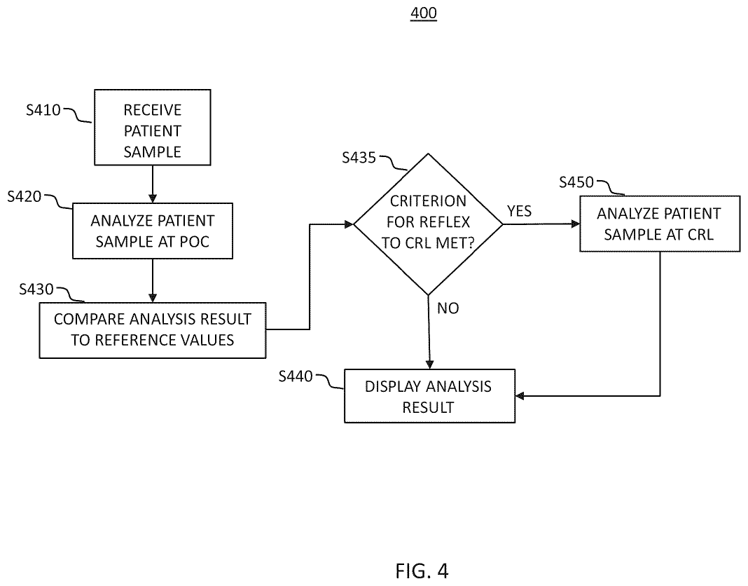

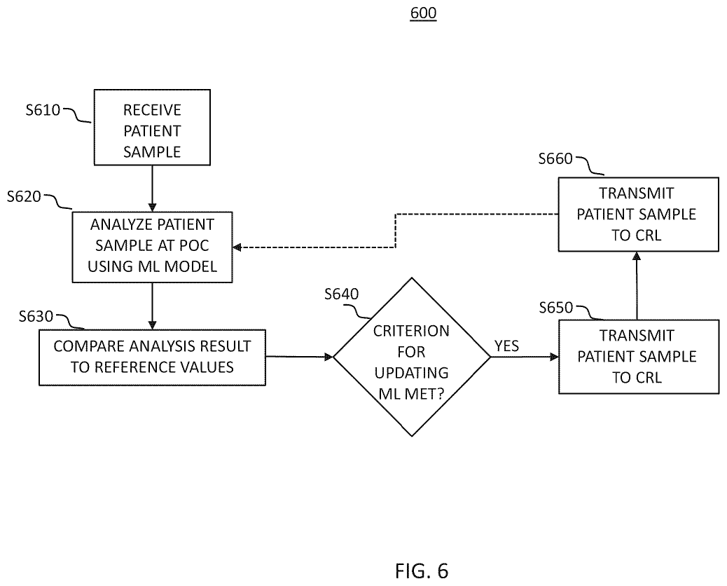

Further still, example methods are described herein with respect to FIG. 4. Such figures are described to include blocks associated with computer-executable instructions. It should be noted that the respective instructions associated with any such blocks herein need not be separate instructions and may be combined with other instructions to form a combined instruction set. The same set of instructions may be associated with more than one block. In this regard, the block arrangements shown in method FIGS. 4 and 6 herein are not limited to an actual structure of any program or set of instructions or required ordering of method tasks, and such method FIGS. 4 and 6, according to some aspects of the disclosure, merely illustrate the tasks that instructions are configured to perform, for example upon execution by a data processing device system in conjunction with interactions with one or more other devices or device systems.

FIG. 1 schematically illustrates a system 100 according to some aspects of the disclosure. In some aspects of the disclosure, the system 100 may be a computing device 200 (as shown in FIG. 2). In some aspects of the disclosure, the system 100 includes a data processing device system 110, an input-output device system 120, and a processor-accessible memory device system 130. The processor-accessible memory device system 130 and the input-output device system 120 are communicatively connected to the data processing device system 110.

The data processing device system 110 includes one or more data processing devices that implement or execute, in conjunction with other devices, such as one or more of those in the system 100, control programs associated with some of the various aspects of the disclosure. Each of the phrases “data processing device,” “data processor,” “processor,” and “computer” is intended to include any data processing device, such as a central processing unit (“CPU”), a circuit, a field programmable gate array (FPGA), a desktop computer, a laptop computer, a mainframe computer, a tablet computer, a personal digital assistant, a cellular phone, and any other device configured to process data, manage data, or handle data, whether implemented with electrical, magnetic, optical, biological components, or the like.

The memory device system 130 includes one or more processor-accessible memory devices configured to store information, including the information needed to execute the control programs associated with some of the various aspects of the disclosure. The memory device system 130 may be a distributed processor-accessible memory device system including multiple processor-accessible memory devices communicatively connected to the data processing device system 110 via a plurality of computers and/or devices. On the other hand, the memory device system 130 need not be a distributed processor-accessible memory system and, consequently, may include one or more processor-accessible memory devices located within a single data processing device.

Each of the phrases “processor-accessible memory” and “processor-accessible memory device” is intended to include any processor-accessible data storage device, whether volatile or nonvolatile, electronic, magnetic, optical, or otherwise, including but not limited to, registers, floppy disks, hard disks, Compact Discs, DVDs, flash memories, ROMs (Read-Only Memory), and RAMs (Random Access Memory). In some aspects of the disclosure, each of the phrases “processor-accessible memory” and “processor-accessible memory device” is intended to include a non-transitory computer-readable storage medium. In some aspects of the disclosure, the memory device system 130 can be considered a non-transitory computer-readable storage medium system.

The phrase “communicatively connected” is intended to include any type of connection, whether wired or wireless, between devices, data processors, or programs in which data may be communicated. Further, the phrase “communicatively connected” is intended to include a connection between devices or programs within a single data processor, a connection between devices or programs located in different data processors, and a connection between devices not located in data processors at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some aspects of the disclosure.

The input-output device system 120 may include a mouse, a keyboard, a touch screen, another computer, or any device or combination of devices from which a desired selection, desired information, instructions, or any other data is input to the data processing device system 110. The input-output device system 120 may include any suitable interface for receiving information, instructions or any data from other devices and systems described in various ones of the aspects of the disclosure.

The input-output device system 120 also may include an image generating device system, a display device system, a speaker device system, a processor-accessible memory device system, or any device or combination of devices to which information, instructions, or any other data is output from the data processing device system 110. In this regard, if the input-output device system 120 includes a processor-accessible memory device, such memory device may or may not form part or all of the memory device system 130. The input-output device system 120 may include any suitable interface for outputting information, instructions or data to other devices and systems described in various ones of the aspects of the disclosure. In this regard, the input-output device system may include various other devices or systems described in various aspects of the disclosure.

FIG. 2 shows an example of a computing device system 200, according to some aspects of the disclosure. The computing device system 200 may include a processor 250, corresponding to the data processing device system 110 of FIG. 1, in some aspects of the disclosure. The memory 251, input/output (I/O) adapter 256, and non-transitory storage medium 257 may correspond to the memory device system 130 of FIG. 1, according to some aspects of the disclosure. The user interface adapter 254, mouse 258, keyboard 259, display adapter 255, and display 260 may correspond to the input-output device system 120 of FIG. 1, according to some aspects of the disclosure. The computing device 200 may also include a communication interface 252 that connects to a network 253 for communicating with other computing devices.

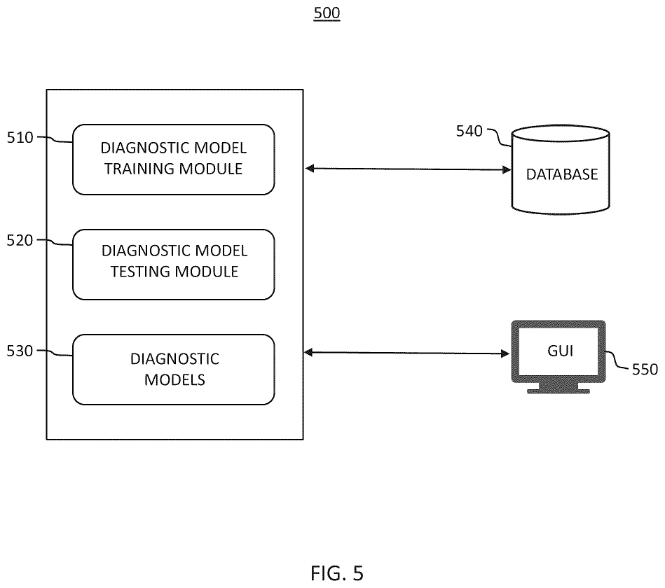

Referring to FIGS. 1, 2, 4, and 6 various methods 400 and 600 may be performed by way of associated computer-executable instructions according to some example aspects of the disclosure. In various example aspects of the disclosure, a memory device system (e.g., memory device system 130) is communicatively connected to a data processing device system (e.g., data processing device systems 110, otherwise stated herein as “e.g., 110”) and stores a program executable by the data processing device system to cause the data processing device system to execute various aspects of methods 400 and 600 via interaction with at least, for example, various databases 540 shown in FIG. 5. In these various aspects of the disclosure, the program may include instructions configured to perform, or cause to be performed, various ones of the instructions associated with execution of various aspects of methods 400 and 600. In some aspects of the disclosure, methods 400 and 600 may include a subset of the associated blocks or additional blocks than those shown in FIGS. 4 and 6. In some aspects of the disclosure, methods 400 and 600 may include a different sequence indicated between various ones of the associated blocks shown in FIGS. 4 and 6.

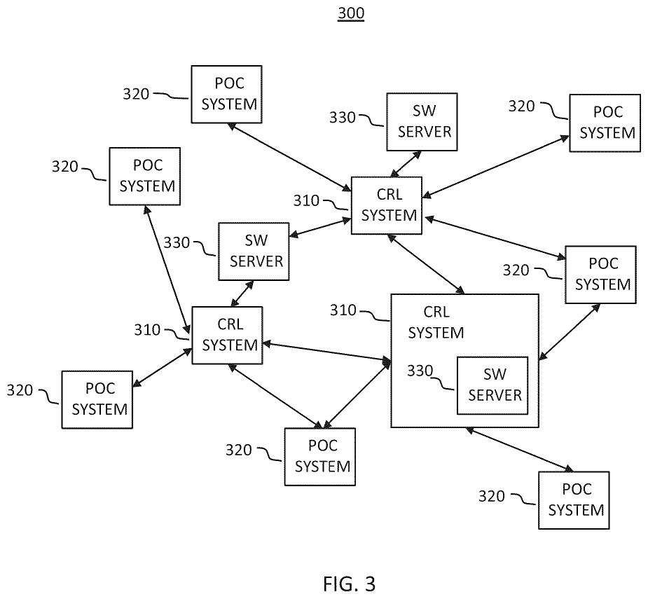

FIG. 3 is a block diagram of a hybrid medical diagnostic system 300 according to various aspects of the disclosure. In some aspects of the disclosure, the hybrid medical diagnostic system 300 includes one or more CRL systems 310, one or more POC systems 320, and one or more software servers 330. The software servers 330 may be connected to the CRL systems 310 or be implemented within the CRL systems 310. In some aspects of the disclosure, the software servers 330 provide centralized software development resources and support for generating medical diagnostic algorithms using machine learning models. In some aspects of the disclosure, one or more POC systems 320 may be connected to one or more CRL systems 310. In some aspects of the disclosure, patient sample data collected at the POC systems 320 is transmitted to the CRL systems 310 for advanced analyses, and the results are transmitted back to the POC systems 320.

In conventional POC systems, one approach is for a POC system 320 to collect data about a patient sample, using various tests and observations, and analyze it with one or more algorithms that can be executed using the limited computing power of the POC system 320, or on a processing module attached to the POC system 320. These algorithms analyze the data from the patient sample using pre-trained machine learning models and report the results to the user (e.g., a clinician). This approach can be utilized in, for example, POC systems for hematology, clinical chemistry, immunoassay, urinalysis, coagulation, blood gases, and electrolytes. The POC diagnostic process may include the following steps: drawing/collecting a patient sample, manually or automatically analyzing the sample using an analyzer to generate data, using the data, along with other observations, as input to various medical diagnostic algorithms that interpret the data, and reporting the outputs from the medical diagnostic algorithms to the clinician. In some examples, patient samples are not drawn or collected, but the patient or portions of the patient are imaged directly, for example in the case of X-ray radiography, computed tomography, and the like. In these circumstances, the diagnostic process may include imaging the patient or portions of the patient, manually or automatically analyzing the images using an analyzer to generate data, using the data, along with other observations, as input to various medical diagnostic algorithms that interpret the data, and reporting the outputs from the medical diagnostic algorithms to the clinician.

A POC system for image recognition of cell morphology may include one or more of the following processing steps: obtaining the image data of samples to be analyzed by capturing images, for example, using microscopy techniques; cleaning and preprocessing the acquired images to enhance the quality and remove any noise or artifacts (for example, using resizing, cropping, denoising, and normalization); segmenting the structures of interest from the background or other structures using various segmentation techniques such as thresholding, edge detection, region growing, or machine learning-based methods; extracting relevant features, such as shape, texture, intensity, or spatial properties, from the segmented structures using morphological operations, statistical analysis, and image texture analysis; and inputting the extracted features into a machine learning-based POC system to obtain, as output from the POC system, classification data.

The specific classification performed by the machine learning-based POC system for morphology analysis depends on the diagnostic objective and the types of structures being analyzed. Some examples of common classifications in medical diagnostics include:
Normal vs. abnormal cells: The model distinguishes between normal cells and cells exhibiting abnormal morphology, such as cancerous or diseased cells. This classification helps identify potential pathological conditions.
Morphologic cell assessment:
   Detection of overall presentation of cell; and
   Detection of cell objects and features, such as nuclear features, vacuoles, organisms, inclusions, and the like.
Cell type identification: The model classifies cells into different cell types based on their morphology and/or size. For example, in blood cell analysis, the model can classify red blood cells, white blood cells, and platelets.
Subcellular component identification: The model identifies and classifies specific subcellular components or organelles within cells. This can include classifying nucleus, cytoplasm, mitochondria, or other cellular structures.
Differentiation of cell stages: The model determines the stage or maturation level of cells.
Parasite detection: The model detects eggs, ova, parasites, bacteria, or the like in a sample, such as an car swab sample or fecal sample.
Disease-specific classification: The model classifies cells based on specific diseases or conditions. For example, in histopathology, the model can classify cells based on different types of cancer.

The algorithms residing in the POC system 320 may utilize pre-trained machine learning models to analyze the data and provide diagnostic information. Generally, these machine learning models are developed (trained and tested) at centralized locations (for example, such as the CRL systems 310 or software development companies that deploy the trained models into the software servers 330), that collect large amounts of patient data from multiple sources (including, for example, multiple POC systems 320) and have extensive computing resources to perform the necessary training and testing to generate the machine learning models. One drawback of the purely POC system based diagnostic approach is that the machine learning algorithms deployed at the POC systems 320, which have limited computing resources, cannot leverage continuous learning technologies to continually refine and update the machine learning models as new patient sample data is received. Moreover, each POC location is only going to see a limited population of patients. Thus, any incremental or continuous learning performed at the POC system 320 would suffer from potentially biased or sparse data. In contrast, the CRL system 310 receives aggregate data from a number of POC systems 320, and is more suited to execute the incremental and continuous learning algorithms to refine the trained machine learning models.

In classical machine learning, an algorithm has access to all training data at the same time. In incremental/continual learning, however, the data instead arrives in a sequence, or in a number of steps, and the underlying distribution of the data changes over time. Incremental learning is a powerful technique to allow the machine learning models to evolve and improve with time, using newly acquired data from field use. This process more closely resembles human learning and knowledge acquisition. Human beings continually learn new concepts, and change previously learned concepts, as they acquire more knowledge. However, conventional machine learning algorithms rely on a predefined static collection of data (knowledge) to infer and predict the desired information in a field-use setting, which severely limits their performance when the underlying concepts change over time. Incremental/continuous learning techniques need to handle concept drift—the change in the relationships between input and output data in the underlying problem over time.

Using new sample data to retrain the machine learning algorithms can alleviate the problem of concept drift. In cases where such drift is desired, an important problem in machine learning is enabling the trained models to incrementally learn from new streams of data without compromising their performance. For example, when neural networks are trained on sample data from a new task or data distribution, they tend to rapidly lose previously acquired capabilities (predictive or diagnostic power), a phenomenon referred to as catastrophic forgetting. In stark contrast to machine learning, humans can incrementally learn new skills without compromising those that were already learned.

In a conventional purely-CRL system based diagnostic approach, the collected patient samples or data are directly sent to the CRL system 310 for analysis. The sample and/or data is analyzed at the CRL system 310 on automated analyzers, by manual processes, and/or by manual microscopy preparations that are later analyzed by pathologists, to generate diagnostic data. The diagnostic data is used as input to diagnostic algorithms, including advanced machine learning models, to generate results that are then provided back to the clinician at the POC location. In some cases, predefined conditions associated with the automated analysis methods at the POC system 320 may trigger a follow-on test requiring a manual analysis of the sample or sample data by a pathologist (at a CRL system 310) due to morphologic abnormalities, flags, or out of reference interval values. The purely CRL system based diagnostic approach is commonly well received in the medical community due to the professional nature of the laboratory processes and advanced training of the pathologists working with/at CRL systems 310.

However, significant drawbacks of the purely CRL system based diagnostic approach include delay in results due to the time required to transport the samples to the CRL systems 310, the time for board-certified staff to analyze samples and/or data, and increased overhead and cost to support lab technicians and board-certified staff. In some circumstances, such as in the veterinary practice, patients may be brought to a POC location for a diagnostic or wellness visit. If a purely CRL diagnostic approach is utilized, the results may not be available when the patient is still at the POC location, requiring the veterinarian or veterinarian technician to contact the patient's owner later to provide the results and/or treatment plan. This delay in time and inability to provide results and treatment plan while the patient is at the POC can have a significant impact on patient engagement and adherence to treatment protocols.

For some types of samples, the POC system user could prepare the sample by traditional means and then scan the sample on a digital scanner. These digitized samples could be electronically sent to the pathologist at the CRL system 310, eliminating the need for traditional means of sending physical samples to the CRL system 310. Digital sample preparation, however, does not alleviate the time consuming and error prone process of preparing samples for the microscope, or the delay in the provision of results and treatments to the patient.

A purely CRL system based diagnostic approach is compatible with the use of incremental learning to continually refine and improve the machine learning models based on the data received from the POC systems 320. However, as the number of POC systems 320 can be very large, it can become very costly and infeasible to send all the patient sample data collected at the POC systems 320 to the CRL system 310. Thus, a new approach to continuous learning is needed, where only the data that is important, relevant, and helpful in further refining the learned concepts is identified at the POC systems 320 for transmission to the CRL system 310. In some aspects of the disclosure, POC systems 320 consider a number of different factors to determine what data to transmit to the CRL system 310 for continuously/incrementally refining the machine learning models. In some aspects of the disclosure, the POC systems 320 filter the new patient sample data collected at the POC based on its relevance to the diagnostic tasks performed by the machine learning models. If certain data samples are more representative or pertinent to the diagnostic problem, the POC systems 320 can prioritize sending those data samples to the CRL system 310. This helps ensure that the CRL system 310 receives data that is most informative for continuously refining the model.

Further, to ensure that the trained machine learning models remain well-generalized, in some aspects of the disclosure, the POC systems 320 use various sampling techniques to select and transmit a diverse range of data, which covers different variations and scenarios, to the CRL system 310. This diversity can help the model learn robust patterns and reduce bias. By selecting data that spans different regions, demographics, or conditions, the client nodes contribute to a more comprehensive and inclusive training set. The sampling techniques include random sampling, stratified sampling, cluster sampling, importance sampling, uncertainty sampling, and active learning, as examples.

Random sampling involves randomly selecting data instances from the sample data collected at the POC system 320. This technique is simple and cost effective, generally ensures that the selected subset is a representative sample of the overall data distribution and can be implemented by randomly selecting a fixed number of instances or by specifying a percentage of the collected sample data collected at the POC system 320 to be transmitted to the CRL system 310. Stratified sampling is useful when the class distribution of the data sent from the POC system 320 to the CRL system 310 needs to be maintained. Each class or category is proportionally represented in the data sent from the POC system 320 to the CRL system 310, to ensure diversity of the data. This technique is particularly important when dealing with imbalanced datasets where some classes, for example rare or uncommon diseases or presentations, have significantly fewer samples than others. For example, in the veterinary context, blood work may be taken from patients as a matter of course during a wellness visit (depending on the patient's age, breed, etc.), such that robust datasets for blood samples may be available. By contrast, fine needle aspirate (FNA) samples for diagnosing abnormal tissue may be taken from patients primarily in response to detection of abnormal masses (e.g., lumps and bumps), and available datasets for FNA samples may be significantly more limited as compared to blood samples.

Cluster sampling involves partitioning the data into clusters based on certain criteria (e.g., geographic location, demographics, or other relevant factors). Instead of sampling individual instances of patient data collected at the POC system 320, entire clusters are selected as representative data subsets to be transmitted from the POC system 320 to the CRL system 310. This can be beneficial when clusters represent distinct groups or subpopulations within the data, such as different species of animals in veterinary diagnostic systems. Importance sampling assigns weights to each data sample based on its importance or relevance to the diagnostic task. Data samples that are more informative or challenging can be given higher weights, while less informative samples receive lower weights. This technique allows prioritizing important data samples for reflexing to the CRL system 310, enhancing the overall incremental/continuous learning process. Uncertainty sampling focuses on selecting data samples that the current model is uncertain about or finds challenging to classify. By selecting these data samples, the diagnostic system actively targets areas of the data where the trained machine learning model needs further improvement. Common uncertainty sampling strategies include selecting instances with the highest prediction entropy, margin, or confidence scores. Active learning is an iterative sampling approach where the trained machine learning model interacts with the POC systems 320 to select the most informative data samples for further training. The model can query the POC systems 320 for instances it is uncertain about or instances that are expected to have the most impact on improving its performance. This interactive process helps optimize the data selection based on the evolving needs of the machine learning model.

In some aspects of the disclosure, one or more of the above-discussed sampling techniques may be used to determine which data samples are reflexed to the CRL system 310 based on the desired outcomes and/or goals. In some aspects of the disclosure, the POC systems 320 evaluate the quality and reliability of the data sample before sending it to the CRL system 310. Filtering out noisy or erroneous data can prevent the machine learning model from learning from misleading or incorrect information. Data quality checks can involve assessing missing values, outliers, inconsistencies, or known data collection issues. Considerations of privacy and security can be important when transmitting data from the POC systems 320 to the CRL systems 310, for example in human health applications. In some aspects of the disclosure, the POC systems 320 filter out identifying, sensitive, or confidential data that should not be shared. Privacy-preserving techniques, such as data anonymization, encryption, or differential privacy, can be applied to protect individual data privacy while still contributing to the training process.

Continuous learning is important because newly acquired sample data at the POC systems 320 can capture the latest trends, changes, or patterns, enabling the trained machine learning models to adapt to evolving conditions. However, it's important to strike a balance between including fresh data and maintaining consistency with historical data to avoid concept drift or model instability.

In some aspects of the disclosure, a mixed POC-CRL system based diagnostic approach permits a dual-level diagnostic method, where the majority of patient samples are analyzed at the POC systems 320 but at least some of the data generated by the POC system 320 may be transmitted (reflexed) to a CRL system 310 for expert analysis and further use. The mixed POC-CRL system based diagnostic approach balances cost and time with advanced diagnostics and accuracy. Cheaper and faster diagnostic services are provided at the POC systems 320, whenever possible. If the diagnostic information provided at the POC systems 320 is not accurate, or not available/possible, the patient data is reflexed to the CRL system 310 for more accurate and advanced diagnosis-albeit at an increased cost.

Some use cases for this type of mixed POC-CRL diagnostic analysis are cardiology, digital radiography, and digital microscopy. With digital radiography, the X-ray source and digital capture systems are present at the POC system 320 at the point-of-care. The digital images are either reviewed by a trained practitioner at the point-of-care, if available, or sent to a radiologist at a central reference laboratory for analysis. The practitioner or radiologist may use algorithms, and advanced machine learning models, to provide additional diagnostic information, including cardiology information like a vertebral heart score.

Cardiology includes measuring heart signals of the patient onsite. The ECG may be available for viewing at the POC system 320, if the clinician wants and is able to interpret it, but it can also be sent to the CRL system 310 for cardiologist review and reporting. POC systems 320 may include automated algorithms that can screen ECGs and reflex abnormal data to a CRL system 310 for cardiologist review, while automatically responding to normal data. This hybrid approach promotes efficiency and cost effectiveness, limiting expensive CRL-based diagnostic testing to only those samples that show some potential problems, while handling all normal samples cheaply using the POC systems 320 present at the point of care.

Similarly, digital microscopy can be used to capture digital images of samples (e.g., blood, FNA, or the like) and provide them to clinical pathologists at a CRL system 310, who interpret them and provide diagnostic information back to the clinician at the point of care. Machine learning algorithms can be implemented to interpret the digital images within the POC system 320.

The mixed (hybrid) approach permits the POC system 320 to capture data from the samples and analyze the data with the currently released software that resides on the POC system 320. A follow-on analysis is performed by algorithms on the POC system 320 or as a cloud service to determine if additional information is available by other means that are not yet available to customer on the POC system 320. If additional information is determined to be available, the customer can choose to send the digital data packet from the system to a CRL 310 or to a server location 330, where additional analyses can be performed. These analyses can include manual review of the data package by a trained professional, additional software tool analyses that are then reviewed by a trained professional, and/or additional software tools that provide autonomous information.

In some instances, these additional software tools and manual analyses may have been fully validated for diagnostic use in POC systems 320, but their deployment to the POC system 320 is lagging due to a number of factors including software development time, software validation, and the capability to deploy the software onto the limited computing resources existing on the POC systems 320. The hybrid approach provides the customer with additional information than they could get waiting for the conventional process of deploying new software to the POC system 320. In some aspects of the disclosure, this hybrid approach also provides alternative implementations where the POC system 320 has a set of onboard algorithms that provide the preliminary and immediate information, but other algorithms that do not lend themselves to onboard processing can be implemented at the CRL system 310 and provide augmented analyses to the customer in an automated manner.

Further, the POC systems 320 may include predefined conditions that trigger transmission of patient data to a CRL system 310 for further analysis. For example, if the diagnostic models at the POC system 320 show that the patient sample data may indicate an abnormal or unexpected result or that the patient sample is a sample type of specific interest, the patient sample data may be transmitted to the CRL system 310 for further analysis and verification of the result. Similarly, if the diagnostic models provided at the POC system 320 cannot provide a result, or the accuracy of the prediction/result is below a predefined threshold, the patient sample data may be transmitted to the CRL system 310 for further analysis and verification of the result.

Various aspects of the hybrid diagnostic system 300 may be realized by software, or more precisely, an application program running on a microprocessor, or by firmware or hardware implementing the program on the CRL systems 310, and POC systems 320. The hybrid diagnostic system 300 may include one or more memories which store various data and program modules associated with the diagnostic methods.

FIG. 4 is a high-level flowchart of a method 400 of a hybrid approach to patient diagnosis. In step S410, a patient sample is received or collected at the point of care. In step S420, the patient sample is analyzed using one or more POC systems 320 at the point of care. In step S430, the analyzed results are compared to reference values to determine whether one or more criteria or predefined conditions for advanced analyses at a CRL system 310 have been met. If a criterion or predefined condition for advanced analysis is met (“YES” at step S435), the patient sample data is reflexed (sent) to the CRL system 310 for further analysis. If none of the criteria or predefined conditions for advanced analyses are satisfied (“NO” at step S435), the results of the local analysis performed at the POC system 320 are displayed to the user in step S440. If the patient sample data has been reflexed to the CRL system 310 (“YES” at step S435), the sample data is further analyzed by pathologists and/or using advanced algorithms available at the CRL system 310 in step S450. In some examples, the results of the advanced analyses are transmitted back to the POC system 320 and displayed to the user in step S440. In some examples, the patient sample data is reflexed to the CRL system 310 for further analysis and/or algorithm development, but the results of the local analysis performed at the POC system 320 are displayed to the user in step S440. In some embodiments, the results of the advanced analyses are transmitted back to the POC system 320, and the results of the advanced analyses and the results of the local analysis performed at the POC system are both displayed to the user in step S440.

There are two important considerations when selecting data samples to be reflexed to the CRL system 310—(1) increasing the diagnostic performance/ability for a particular data sample and (2) improving the overall diagnostic performance of the trained machine learning models in the system.

In some cases, the algorithms available at the POC system 320 may be unable to provide a diagnosis based on the data samples obtained from the patient, either because the output from the available at the POC system 320 on the specific data sample has a low uncertainty or confidence or there are no validated algorithms available to the POC system 320 to analyze the data sample. For example, the algorithms used for diagnostic discrimination may be species specific, and while there may be an algorithm available at the POC system 320 to analyze a particular type of data sample obtained from one species (say, a dog), a similar algorithm may be unavailable for another species (say, a camelid).

Conversely, the diagnostic output from the algorithms available at the POC system 320 may have a high confidence value or certainty associated with the model prediction, but may be nevertheless suspect because there are few or no similar cases in the data set or only a small percentage of the cells in the data sample may be identified as relevant to the diagnostic prediction. In these cases, the data sample is reflexed to the CRL system 310 for advanced analysis or pathologist review, which can provide fast diagnosis of the underlying medical conditions.

In some cases, the data sample may have originated from a known patient with an existing medical condition for which other data exists. For example, the data sample may have been obtained from a lesion on a subject that has other lesions for which data samples were previously collected. Or, the data sample may have been obtained from a same lesion as another data sample, but with a different presentation (one side hard/one side soft, etc.) In these instances, it may be beneficial to reflex the data samples to a CRL system 310 for pathologist review, to fully consider all data related to a same medical condition comprehensively, and/or to resolve potentially conflicting diagnostic information in the data samples. For similar reasons, it may also be beneficial to reflex data samples that are important to the diagnosis, hard to obtain (for example, from fine needle aspiration or body cavity fluids), or indicative of a rare condition to a CRL system 310 for pathologist review. Data samples that suggest, at the POC system 320, a diagnosis that would lead to severe treatment or euthanasia may also be reflexed to the CRL system 310 for further confirmation of the severity of diagnosis.

In some cases, the data sample may be reflexed to the CRL system 310 because of customer instructions or to provide a comparison of the algorithm output with a pathologist answer to confirm/highlight correspondence between the two, even though reflexing the data sample to the CRL system 310 may incur additional costs/expense to the customer. Data samples may also be reflexed to the CRL system 310 to support or build case studies corresponding to worst case scenarios or advanced stage presentations of a medical condition, to identify representative data samples for a medical condition, or based on keywords or medical notes that may be associated with the patient from who the data samples were obtained.

Even though, in the above discussed cases, the data samples are being reflexed to the CRL system 310 to provide improved diagnosis or confirmation of the underlying medical conditions, the reflexed data samples can also be used as new training data to improve the performance of existing algorithms at both the POC system 320 and the CRL system 310, or train new algorithms to discriminate diagnostic information represented in the reflexed data samples.

In some aspects of the disclosure, some data samples are reflexed to the CRL system 310 specifically to be used as new/additional training data to continually retrain and improve the performance of the algorithms residing on the POC system 320 and the CRL system 310, or train new algorithms to discriminate diagnostic information represented in the reflexed data samples. For example, data samples that have a low confidence or certainty associated with the predicted output from the algorithms may be reflexed to the CRL system 310 to be used to further train the algorithms. The POC system 320 may also reflex data samples that are important to the diagnostic prediction, data samples that correspond to sparse distributions in the training data set, and data samples obtained from patients for which automated algorithms are not available at the POC system 320 to the CRL system 310 for use as new/additional training data. In some aspects of the disclosure, the POC system 320 may reflex data samples at predefined periodic intervals to capture any changes in the trends learned by the models, and to capture new trends.

FIG. 6 is a high-level flowchart of a method 600 of updating the machine learning models using new data samples received or generated at the POC system 320 and reflexed to the CRL 310. In step S610, new patient data samples are received or generated at the point of care. In step S620, the data sample is analyzed using one or more POC systems 320 at the point of care. In step S630, the analyzed results are compared to reference values to determine whether one or more criteria for reflexing the data sample to a CRL system 310 for updating the machine learning models have been met. If a criterion for using the data sample as part of new training data to update the machine learning models is met (“YES” at step S640), in step S650, the patient sample data is reflexed (sent) to the CRL system 310 for use as new training data. In step S660, continuous learning techniques, discussed herein, are executed to update the machine learning models using the new sample data. The updated machine learning models are deployed back to the POC system 320, as appropriate.

Referring to FIGS. 3 and 5, in some aspects of the disclosure, the hybrid diagnostic system 300 executes a software-based clinical diagnostic system 500 on one or more of the CRL systems 310, POC systems 320, and software servers 330. The clinical diagnostic system 500 includes a diagnostic model training module 510, a diagnostic model validation/testing module 520, and one or more diagnostic models 530. In some aspects of the disclosure, the clinical diagnostic system 500 may be communicatively connected to one or more databases 540. In some embodiments, the clinical diagnostic system 500 includes a graphical user interface 550 to permit a user to interact with the system. In some embodiments, the one or more diagnostic models 530 may be stored in a database 540. In some aspects of the disclosure, the diagnostic machine learning models 530 take patient sample data as inputs and provide diagnostic information as outputs.

In some aspects of the disclosure, the machine learning models 530 are trained and validated/tested on a large collection of annotated patient sample data, stored in one or more databases 540, using the diagnostic model training module 510 and the diagnostic model validation/testing module 520. In some aspects of the disclosure, the diagnostic model training module 510 receives the patient sample data and extracts features to be used as inputs for training machine learning models 530 to perform classification. The ground truth (target output from the machine learning models 530) is provided by the diagnosis associated with the corresponding set of tests and observations in the patient sample data.

In some aspects of the disclosure, one or more machine learning models 530 are generated and maintained at the CRL systems 310 or software servers 330 and used for classification of new patient data at CRL systems 310 and POC systems 320.

In a training phase, a set of features is extracted from a collection of patient sample data and provided as training data to one or more machine learning models, such as neural networks, as inputs. The machine learning models learn the patterns present in the data they are given and use an error between the expected and actual output to correct themselves by adjusting their parameters as more data is input (for example, by correcting the weights and biases for each connected pair of neurons in a neural network). The expected outcome can be provided by annotated ground truth data associated with each patient sample. In some embodiments, validation and testing of the trained machine learning models is performed to ensure that the models are generalized (they are not overfitted to the training data and can provided similar performance on new data as on the training data).

In some aspects of the present disclosure, a portion of the patient sample data is held back from the training set for validation and testing. The validation dataset is used to estimate the machine learning model's performance while tuning the model's parameters (such as the weights and biases of a neural network). The test dataset is used to give an unbiased estimate of the performance of the final tuned machine learning model. It is well known that evaluating the learned model using the training set would result in a biased score as the trained model is, by design, built to learn the biases in the training set. Thus, to evaluate the performance of a trained machine learning model, one needs to use data that has not been used for training.

In some aspects of the disclosure, the collected patient sample data set can be divided equally between the training set and the testing set. The machine learning models are trained using the training set and their performance is evaluated using the testing set. The best performing machine learning model may be selected for use. The machine learning model is considered to be generalized or well-trained if its performance on the testing set is within a desired range of the performance on the training set. If the performance on the test set is worse than the training set (the difference in error between the training set and the testing set is greater than a predefined threshold), a two-stage validation and testing approach may be used.

In some aspects of the disclosure, in a two-stage validation and testing approach, the collected patient sample data set is divided between the training set, the validation set, and the testing set. The machine learning models are first trained using the training set, then their parameters are adjusted to improve their generalization using the validation set, and, finally, the trained machine learning models are tested using the testing set. The patient sample data set may be divided equally between the desired training, validation, or testing sets. This works well when there is a large collection of data to draw from. In cases where the collection of data samples is limited, other well-known techniques, such as leave one out cross validation and testing or k-fold cross validation may be used to perform validation and testing. Cross-validation is a resampling procedure used to evaluate machine learning models on a limited data set. The procedure has a single parameter called k that refers to the number of groups that a given data set is to be split into. As such, the procedure is often called k-fold cross-validation. When a specific value for k is chosen, such as k=10, the procedure becomes 10-fold cross-validation.

Cross-validation is primarily used to estimate how the trained model is expected to perform in general when used to make predictions on data not used during the training of the model. The dataset is shuffled randomly and divided into a predefined number (k) of groups. The training and testing process is performed k times, with one of the groups of data being held out as the testing set for each iteration and the remaining k−1 groups being used as the training set. Each model is fitted (trained) on the training set and evaluated (tested) on the test set to determine the level of generalization of the trained models.

In addition to preventing overfitting, k-fold cross validation can also help determine the model structure and the parameter training process for the machine learning model. For example, a neural network model can have one or more “hidden” layers of neurons between the input layer and the output layer. Further, different neural network models can be built with different numbers of neurons in the hidden layers and the output layers. In some aspects of the disclosure, in the training phase, a plurality of machine learning models (for example, neural network models having different numbers of layers and different numbers of neurons in each layer) are generated. Each of the plurality of machine learning models is trained using k-fold cross validation, resulting in a score that predicts the skill of each model in providing the correct expected output. The model (for example, number of layers and number of neurons in each layer of a neural network) having the highest predictive score is selected and then trained, using a larger portion of the patient sample data to generate the final machine learning model.

In some aspects of the disclosure. the machine learning model is a convolution neural network. Convolutional neural networks (CNNs) are a type of deep learning model specifically designed for processing and analyzing visual data, such as medical images. Inspired by the human visual system, CNNs utilize convolutional layers to extract local patterns and hierarchical representations from the input data. This ability to automatically learn and recognize intricate features makes CNNs particularly suitable for medical image classification tasks.

The architecture of a CNN comprises several layers, each serving a specific purpose in the classification process. The primary layers in a typical CNN architecture for medical diagnostics are convolution layers, pooling layers, activation functions, and fully connected layers. Convolutional layers perform convolution operations using learnable filters, detecting local patterns and features in the medical data. By capturing information at multiple scales, CNNs can identify important structures and abnormalities. Pooling layers reduce the spatial dimensions of the feature maps obtained from convolutional layers. Common pooling techniques, such as max pooling, downsample the feature maps while retaining the most salient information. This spatial reduction helps reduce computational complexity and enhances translation invariance. Activation functions introduce non-linearity to the network, enabling CNNs to model complex relationships within the medical data. Popular activation functions include ReLU (Rectified Linear Unit) and sigmoid, which enhance the network's ability to learn discriminative features. Fully connected layers connect all neurons from the previous layer to every neuron in the subsequent layer. These layers integrate the learned features and make the final classification predictions. In medical diagnostics, the output layer typically represents the different disease classes or diagnostic outcomes.

The training of CNNs involves two key processes: forward propagation and backpropagation. During forward propagation, the input medical data passes through the layers of the network, and the predictions are generated. These predictions are then compared with the ground truth labels to calculate the loss. Backpropagation involves calculating the gradients of the loss function with respect to the network's parameters and adjusting those parameters using optimization algorithms such as stochastic gradient descent (SGD) or Adam. The process of forward propagation and backpropagation is iteratively repeated on a training dataset until the network learns to accurately classify medical data.

CNNs excel in analyzing medical data such as X-rays, CT scans, and MRIs, aiding radiologists in the diagnosis of various conditions. By learning from extensive datasets, CNNs can detect patterns associated with diseases like lung cancer, brain tumors, fractures, and abnormalities in different organs. In histopathology, CNNs have proven invaluable for identifying cancerous cells and tissue patterns. By analyzing digitized histopathology slides, CNNs can assist pathologists in detecting and classifying different types and stages of cancer, improving diagnostic accuracy and reducing workload.

The adoption of CNNs in medical diagnostics offers numerous benefits, including improved accuracy, time efficiency, and clinician assistance. CNNs have consistently outperformed traditional machine learning methods and human experts in medical image classification tasks, leading to more accurate diagnoses. CNNs can rapidly analyze large volumes of medical data, providing quick and reliable diagnostic predictions. This efficiency allows healthcare professionals to make timely decisions and provide better patient care. CNNs can serve as powerful tools for healthcare professionals, providing them with additional support and enhancing their diagnostic capabilities. This collaboration between human expertise and AI can lead to improved patient outcomes.

Machine learning models according to the present disclosure are not limited to neural networks, and any suitable other or combination of other machine learning models, such as a Markov random field network, support vector machine, random forest of decision trees, or k-nearest neighbor, or the like may be used to provide diagnostic information from patient sample data.

In some aspects of the disclosure, the trained machine learning models 530 are refined over time, using continuous learning techniques, as new patient sample data is collected at the POC systems 320. Continuous learning is the ability of a trained machine learning model to autonomously learn and adapt in field use (production) as new data comes in. Continuous learning mimics the human ability to continually acquire, fine-tune, and transfer knowledge and skill throughout a person's lifespan.

Although continuous learning machine learning models may sound ideal for medical purposes, in practice, there are many long-standing challenges in applying them. One main obstacle with continuous learning is catastrophic forgetting (or catastrophic interference phenomenon), in which the new information interferes with what the machine learning model has already learned. This can lead to an abrupt decrease in performance while the new data is being integrated, or even worse, an overwrite of the model's previous knowledge with the new data. Most of the current applications for continuous learning in nonmedical fields are less critically impacted by this limitation. However, the stakes for real-time medical applications of machine learning are high due to their impact on health outcomes.

A simple solution to catastrophic forgetting is to completely retrain the model each time new data is available, but this can be computationally expensive and inhibit real-time inferences. While advances in cloud computing may provide a solution to this problem of computational complexity and cost, the GPU accelerated resources that are needed to retrain on the full datasets are complex to create and are difficult to securely maintain. Moreover, healthcare information governance across different countries is constantly evolving, making it difficult to maintain compliance. In addition, the availability of retrospective training sets needed to fully retrain the model with new data is especially challenging in healthcare due to consent for use constraints. Thus, completely retraining the trained machine learning models on both the old data and the new data may not be feasible.

Furthermore, in the United States, only a few automated algorithms have been approved by the Food and Drug Administration (FDA) for limited capacities such as detection of diabetic retinopathy or breast abnormalities. All of these algorithms have been “locked” for safety, to prevent any potential for further learning or change post-approval. However, continual learning (i.e., “unlocked”) ML models may be more advantageous as they are able to incrementally learn from their mistakes and fine-tune their performance with progressively more data, similar to the ways that human clinicians learn.

There are certain areas within clinical medicine where continual learning ML models could be safely implemented. One example is diagnostic testing, but the labeling of the new data would be a rate-limiting step. When new patient data becomes available, the trained model would perform inference and make a diagnostic prediction. The new data would also need to be manually graded using the reference standard, and the results would then be used to update the model. Manual image grading is a time-consuming step that will limit the overall utility of an automated AI algorithm since all new incremental data will require human input to produce reliable labels, but the performance of the model as it “learns” would not directly affect patient outcomes.

One major problem with continual learning is catastrophic forgetting, which can occur when a trained machine learning model forgets previously learned knowledge while learning new knowledge. Various techniques, such as regularization, rehearsal, dynamic architecture, memory-augment models, and generative replay, may be used to prevent catastrophic forgetting in continual learning.

In some aspects of the disclosure, regularization techniques can be used to prevent overfitting of the trained ML model to the new data. Overfitting occurs when the model becomes too specialized on the new data and forgets the previously learned knowledge. Regularization techniques can be used to penalize complex models that are more likely to overfit. The most commonly used regularization techniques are weight decay, dropout, and early stopping. In the context of a neural network type ML model, weight decay involves adding a penalty term to the function that penalizes large weights. This technique encourages the model to use small weights, which can help prevent overfitting. Dropout is another regularization technique that randomly drops out some of the neurons in a neural network model during training. This technique can help prevent the model from becoming too specialized on the new data. Early stopping is another commonly used regularization technique that stops the training of the model when the performance on the validation set stops improving. This technique prevents the model from overfitting to the new data.

In some aspects of the disclosure, in rehearsal techniques, the trained ML model is retrained on the new data along with some previously training data to prevent forgetting. This can be achieved by storing some of the previous training data and randomly selecting some of it to be used during training on the new data. Rehearsal can be done using several strategies such as random selection, prioritized selection, or intelligent selection. Random selection involves randomly selecting some of the previous training data during retraining with new data. Prioritized selection involves selecting the most important previous data based on some criteria. Intelligent selection involves selecting the previous data that is most relevant to the new data.

In some aspects of the disclosure, the third technique used to prevent catastrophic forgetting is dynamic architecture. Dynamic architecture refers to modifying the architecture of the trained ML model based on the new data to prevent catastrophic forgetting. This can be done by, for example, adding or removing neurons or layers in a neural network ML model based on the new data. The idea is to allow the model to adapt to the new data while preserving the previously learned knowledge. However, modifying the architecture of the model can be computationally expensive and requires careful tuning.

In some aspects of the disclosure, memory-augmented networks are used to incorporate external memory modules that allow the trained ML model to store and retrieve information. This approach can help prevent forgetting by allowing the ML model to explicitly store information about the previously learned tasks. Memory-augmented networks can be divided into two categories: ML models with external memory and ML with internal memory. ML models with external memory include models like Neural Turing Machines (NTMs), Differentiable Neural Computers (DNCs), and Memory Networks (MNs). ML models with internal memory include models like Long Short-Term Memory (LSTM), Gated Recurrent Units (GRUs), and Transformer-based models.

Similar to rehearsal techniques, a fifth technique to prevent catastrophic forgetting, in some aspects of the disclosure, is generative replay. Generative replay involves generating synthetic samples of the previous training data and using them to train the ML model on the new data. This approach has been shown to be effective in preventing forgetting and can be combined with other techniques for better performance. Generative replay can be done using several strategies such as generative adversarial networks (GANs), variational autoencoders (VAEs), or mixture density networks (MDNs). The idea is to generate synthetic samples that are similar to the previous training data and use them to train the model on the new data.

In some aspects of the disclosure, a combination of data sampling techniques, discussed in detail above, is used to determine what data samples should be sent from the POC system 320 to the CRL system 310 for continually updating/refining the machine learning model. For example, random sampling can be used to select a representative sample set to be transmitted from the POC system 320 to the CRL system 310 periodically. This randomly sampled set can be augments with additional samples obtained by cluster sampling or uncertainty sampling to, additionally, boost the machine learning models' performance on uncommon case or for specific classes/sub-classes.

A second issue with continual learning is concept drift. Concept drift refers to the phenomenon where the statistical properties of the data change over time. In the context of continual learning, concept drift can be both good and bad, depending on the specific scenario. One of the main benefits of concept drift is that it allows the trained ML model to adapt to changing environments. For example, in the case of medical diagnosis, the statistical properties of the data can change over time due as viruses mutate and change, causing different symptoms and providing different test results than expected. An ML model that is trained on data from one set of disease symptoms and markers may not perform well on data from another variant of the microbe that causes the disease. By adapting to the changing statistical properties of the data, the model can maintain its performance over time.

Another benefit of concept drift is that it can help the model to generalize better. Generalization refers to the ability of the model to perform well on data that it has not seen before. By training on data that is representative of the current statistical properties of the data, the model can learn to generalize better to new data. This can be particularly important in scenarios where the data is constantly evolving. Concept drift can also be beneficial in situations where the model needs to detect and respond to changes in the environment. For example, in the case of anomaly detection, the model needs to be able to detect when the statistical properties of the data change, indicating the presence of an anomaly. By being able to adapt to the changing statistical properties of the data, the model can improve its ability to detect anomalies.

Concept drift, however, can lead to poor performance on long-term tasks. In the case of continual learning, the goal is to learn multiple tasks over time without forgetting the previously learned knowledge. However, if the statistical properties of the data change too rapidly, the model may not be able to adapt quickly enough, leading to poor performance on long-term tasks Concept drift can also be a challenge when dealing with rare events or anomalies. In some cases, rare events may occur that are significantly different from the previously seen data. If the model has not been exposed to these rare events, it may not be able to detect them. This can be problematic in scenarios where detecting rare events is critical, such as rare diseases.

In some aspects of the present disclosure, concept drift is handled using a combination of sampling techniques, similar to those employed to address catastrophic forgetting. However, unlike catastrophic forgetting, concept drift may be desirable in some cases. Thus, the diagnostic system 300 can selectively sample the data to be reflexed form the POC system 320 to the CRL system 310 based on whether concept drift is desired or not. If concept drift is desirable, because there is an underlying shift in the classification trend, a random sampling of new patient data can adequately capture the shift. If concept drift is not desirable, a stratified sampling approach can be employed to ensure that new data, which may have short term shifts, does not affect the classification models.

Accordingly, it should now be understood that concepts of the present disclosure are directed to methods and systems for performing initial diagnoses using algorithms provided in POC systems but reflexing selected patient data samples, that meet defined criteria, to CRL systems to provide advanced diagnostic capabilities and to permit continuous refinement of the machine learning models present in the POC and CRL systems.

Numbered aspects of the present disclosure are provided below:

In a first aspect A1, the present disclosure provides a method executed by a programmed data processing device system comprising: receiving patient sample data at a point of care; analyzing the received patient sample data at the point of care; evaluating a first result of analyzing the received patient sample data at the point of care to determine whether a criterion for advanced analysis at a central reference laboratory is met; and, in a case where the criterion for advanced analysis is met, reflexing the received patient sample data to the central reference laboratory for the advanced analysis, receiving, at the point of care, a second result of the advanced analysis from the central reference laboratory, and displaying at least one of the first result or the second result on a display at the point of care.

In a second aspect A2, the present disclosure provides the method according to aspect A1, further including, in a case where the criterion for advanced analysis is not met, displaying the first result on the display at the point of care.

In a third aspect A3, the present disclosure provides the method according to any one of aspects A1-A2, further including analyzing the received patient sample data using a machine learning model at the point of care system.

In a fourth aspect A4, the present disclosure provides the method according to aspect A3, wherein the machine learning model is a convolution neural network.

In a fifth aspect A5, the present disclosure provides the method according to any one of aspects A1-A4, further including analyzing the reflexed patient sample data using a machine learning model at the central reference laboratory.

In a sixth aspect A6, the present disclosure provides the method according to aspect A5, wherein the machine learning model is a convolution neural network.

In a seventh aspect A7, the present disclosure provides the method according to any one of aspects A1-A6, further including: receiving a plurality of patient sample data at the central reference laboratory; training a first machine learning model using the plurality of patient sample data; and deploying the trained first machine learning model in the point of care system to analyze the patient sample data received at the point of care.

In an eighth aspect A8, the present disclosure provides the method according to any one of aspects A1-A7, further including: receiving a plurality of patient sample data at the central reference laboratory; training a second machine learning model using the plurality of patient sample data; and analyzing the reflexed patient sample data at the central reference laboratory using the trained second machine learning model.

In a ninth aspect A9, the present disclosure provides the method according to any one of aspects A1-A8, wherein the criterion includes one or more of (i) a case where the first result indicates that the received patient sample data is abnormal, (ii) a case where a difference between the first result and a reference value is greater than a first threshold, (iii) a case where a confidence level associated with the first result is less than a second threshold, (iv) a case where the first result cannot be obtained by analysis at the point of care, or (v) a case where the first result indicates a diagnostic condition that requires advanced analysis at the central reference laboratory.

In a tenth aspect A10, the present disclosure provides a diagnostic system comprising: a memory configured to store instructions; and a processor communicatively connected to the memory and configured to execute the stored instructions to: receive patient sample data at a point of care; analyze the received patient sample data at the point of care; evaluate a first result of analyzing the received patient sample data at the point of care to determine whether a criterion for advanced analysis at a central reference laboratory is met; and in a case where the criterion for advanced analysis is met: reflex the received patient sample data to the central reference laboratory for the advanced analysis, receive, at the point of care, a second result of the advanced analysis from the central reference laboratory, and display at least one of the first result or the second result on a display at the point of care.

In an eleventh aspect A11, the present disclosure provides the system according to aspect A10, wherein the processor is further configured to execute the stored instructions to, in a case where the criterion for advanced analysis is not met, display the first result on the display at the point of care.

In a twelfth aspect A12, the present disclosure provides the system according to any one of aspects A10-A11, wherein the processor is further configured to execute the stored instructions to analyze the received patient sample data using a machine learning model at the point of care system.

In a thirteenth aspect A13, the present disclosure provides the system according to aspect A12, wherein the machine learning model is a convolution neural network.

In a fourteenth aspect A14, the present disclosure provides a hybrid diagnostic system comprising: a first memory configured to store first instructions; a first processor communicatively connected to the first memory and configured to execute the stored first instructions at a point of care to: receive patient sample data at the point of care; analyze the received patient sample data at the point of care; evaluate a first result of analyzing the received patient sample data at the point of care to determine whether a criterion for advanced analysis at a central reference laboratory is met; and, in a case where the criterion for advanced analysis is met: reflex the received patient sample data to the central reference laboratory for the advanced analysis, receive, at the point of care, a second result of the advanced analysis from the central reference laboratory, and display at least one of the first result or the second result on a display at the point of care; a second memory configured to store second instructions; and a second processor communicatively connected to the second memory and configured to execute the stored second instructions at the central reference laboratory to: receive the reflexed patient sample data from the point of care; analyze the received patient sample data at the central reference laboratory to generate the second result; and transmit, to the point of care, the second result of the advanced analysis from the central reference laboratory.

In a fifteenth aspect A15, the present disclosure provides the system according to aspect A14, wherein the first processor is further configured to execute the stored first instructions to, in a case where the criterion for advanced analysis is not met, display the first result on the display at the point of care.

In a sixteenth aspect A16, the present disclosure provides the system according to any one of aspects A14-A15, wherein the first processor is further configured to execute the stored first instructions to analyze the received patient sample data using a machine learning model at the point of care system.

In a seventeenth aspect A17, the present disclosure provides the system according to aspect A16, wherein the machine learning model is a convolution neural network.

In an eighteenth aspect A18, the present disclosure provides the system according to any one of aspects A14-A17, wherein the second processor is further configured to execute the stored second instructions to analyze the reflexed patient sample data using a machine learning model at the central reference laboratory.

In a nineteenth aspect A19, the present disclosure provides the system according to aspect A18, wherein the machine learning model is a convolution neural network.

In a twentieth aspect A20, the present disclosure provides the system according to any one of aspects A14-A19, wherein the second processor is further configured to execute the stored second instructions to: receive a plurality of patient sample data at the central reference laboratory; train a first machine learning model using the plurality of patient sample data; and deploy the trained first machine learning model in the point of care system to analyze the patient sample data received at the point of care.

In a twenty-first aspect A21, the present disclosure provides the system according to any one of aspects A14-A20, wherein the second processor is further configured to execute the stored second instructions to: receive a plurality of patient sample data at the central reference laboratory; train a second machine learning model using the plurality of patient samples; and analyze the reflexed patient sample data at the central reference laboratory using the trained second machine learning model.

In a twenty-second aspect A22, the present disclosure provides a non-transitory computer readable storage medium configured to store a program that executes a diagnostic method, the method comprising: receiving patient sample data at a point of care; analyzing the received patient sample data at the point of care; evaluating a first result of analyzing the received patient sample data at the point of care to determine whether a criterion for advanced analysis at a central reference laboratory is met; and, in a case where the criterion for advanced analysis is met: reflexing the received patient sample data to the central reference laboratory for the advanced analysis, receiving, at the point of care, a second result of the advanced analysis from the central reference laboratory, and displaying at least one of the first result or the second result on a display at the point of care.

In a twenty-third aspect A23, the present disclosure provides a method executed by a programmed data processing device system, the method comprising: receiving patient sample data at a point of care; analyzing the received patient sample data at the point of care using a machine learning model; determining whether a criterion for using the received patient sample data for updating the machine learning model is met based on a result of analyzing the received patient sample data at the point of care; and, in a case where the criterion for updating the machined learning model is met: transmitting the received patient sample data to a central reference laboratory, updating, at the central reference laboratory, the machine learning model by using the patient sample data as new training data for the machine learning mode, and deploying, at the point of care, the updated machine learning model from the central reference laboratory.

In a twenty-fourth aspect A24, the present disclosure provides the method according to aspect A23, wherein the criterion includes one or more of (i) a case where the result indicates that the received patient sample data is relevant to a diagnostic prediction output by the machine learning model, (ii) a case where the result indicates that the received patient sample data is relevant to a diagnostic prediction having sparse training data, or (iii) a case where a confidence level associated with the result is less than a threshold.

In a twenty-fifth aspect A25, the present disclosure provides the method according to any one of aspects A23-A24, further including: defining a sampling strategy for transmitting new training data to the central reference laboratory for updating the machine learning model; and transmitting the received patient sample data to the central reference laboratory in accordance with the sampling strategy.

In a twenty-sixth aspect A26, the present disclosure provides the method according to aspect A25, wherein the sampling strategy includes at least one of random sampling, stratified sampling, cluster sampling, importance sampling, uncertainty sampling, or active learning.

Subsets or combinations of various aspects of the disclosure described above provide further aspects of the disclosure.

These and other changes can be made to the invention in light of the above-detailed description and still fall within the scope of the present invention. In general, in the following claims, the terms used should not be construed to limit the invention to the specific aspects of the disclosure disclosed in the specification. Accordingly, the invention is not limited by the disclosure, but instead its scope is to be determined entirely by the following claims.

1. A method executed by a programmed data processing device system, the method comprising:
receiving patient sample data at a point of care;
analyzing the received patient sample data at the point of care;
evaluating a first result of analyzing the received patient sample data at the point of care to determine whether a criterion for advanced analysis at a central reference laboratory is met; and
in a case where the criterion for advanced analysis is met:
reflexing the received patient sample data to the central reference laboratory for the advanced analysis;
receiving, at the point of care, a second result of the advanced analysis from the central reference laboratory; and
displaying at least one of the first result or the second result on a display at the point of care.

2. The method according to claim 1, further including, in a case where the criterion for advanced analysis is not met, displaying the first result on the display at the point of care.

3. The method according to claim 1, further including analyzing the received patient sample data using a machine learning model at the point of care system.

4. The method according to claim 3, wherein the machine learning model is a convolution neural network.

5. The method according to claim 1, further including analyzing the reflexed patient sample data using a machine learning model at the central reference laboratory.

6. The method according to claim 5, wherein the machine learning model is a convolution neural network.

7. The method according to claim 1, further including:
receiving a plurality of patient sample data at the central reference laboratory;
training a first machine learning model using the plurality of patient sample data; and
deploying the trained first machine learning model in the point of care system to analyze the patient sample data received at the point of care.

8. The method according to claim 1, further including:
receiving a plurality of patient sample data at the central reference laboratory;
training a second machine learning model using the plurality of patient sample data; and
analyzing the reflexed patient sample data at the central reference laboratory using the trained second machine learning model.

9. The method according to claim 1, wherein the criterion includes one or more of (i) a case where the first result indicates that the received patient sample data is abnormal, (ii) a case where a difference between the first result and a reference value is greater than a first threshold, (iii) a case where a confidence level associated with the first result is less than a second threshold, (iv) a case where the first result cannot be obtained by analysis at the point of care, or (v) a case where the first result indicates a diagnostic condition that requires advanced analysis at the central reference laboratory.

10. A diagnostic system comprising:
a memory configured to store instructions; and
a processor communicatively connected to the memory and configured to execute the stored instructions to:
receive patient sample data at a point of care;
analyze the received patient sample data at the point of care;
evaluate a first result of analyzing the received patient sample data at the point of care to determine whether a criterion for advanced analysis at a central reference laboratory is met; and
in a case where the criterion for advanced analysis is met:
reflex the received patient sample data to the central reference laboratory for the advanced analysis;
receive, at the point of care, a second result of the advanced analysis from the central reference laboratory; and
display at least one of the first result or the second result on a display at the point of care.

11. The system according to claim 10, wherein the processor is further configured to execute the stored instructions to, in a case where the criterion for advanced analysis is not met, display the first result on the display at the point of care.

12. The system according to claim 10, wherein the processor is further configured to execute the stored instructions to analyze the received patient sample data using a machine learning model at the point of care system.

13. The system according to claim 12, wherein the machine learning model is a convolution neural network.

14. A hybrid diagnostic system comprising:
a first memory configured to store first instructions;
a first processor communicatively connected to the first memory and configured to execute the stored first instructions at a point of care to:
receive patient sample data at the point of care;
analyze the received patient sample data at the point of care;
evaluate a first result of analyzing the received patient sample data at the point of care to determine whether a criterion for advanced analysis at a central reference laboratory is met; and
in a case where the criterion for advanced analysis is met:
reflex the received patient sample data to the central reference laboratory for the advanced analysis;
receive, at the point of care, a second result of the advanced analysis from the central reference laboratory; and
display at least one of the first result or the second result on a display at the point of care;
a second memory configured to store second instructions; and
a second processor communicatively connected to the second memory and configured to execute the stored second instructions at the central reference laboratory to:
receive the reflexed patient sample data from the point of care;
analyze the received patient sample data at the central reference laboratory to generate the second result; and
transmit, to the point of care, the second result of the advanced analysis from the central reference laboratory.

15. The system according to claim 14, wherein the first processor is further configured to execute the stored first instructions to, in a case where the criterion for advanced analysis is not met, display the first result on the display at the point of care.

16. The system according to claim 14, wherein the first processor is further configured to execute the stored first instructions to analyze the received patient sample data using a machine learning model at the point of care system.

17. The system according to claim 16, wherein the machine learning model is a convolution neural network.

18. The system according to claim 14, wherein the second processor is further configured to execute the stored second instructions to analyze the reflexed patient sample data using a machine learning model at the central reference laboratory.

19. (canceled)

20. The system according to claim 14, wherein the second processor is further configured to execute the stored second instructions to:
receive a plurality of patient sample data at the central reference laboratory;
train a first machine learning model using the plurality of patient sample data; and
deploy the trained first machine learning model in the point of care system to analyze the patient sample data received at the point of care.

21. The system according to claim 14, wherein the second processor is further configured to execute the stored second instructions to:
receive a plurality of patient sample data at the central reference laboratory;
train a second machine learning model using the plurality of patient samples; and
analyze the reflexed patient sample data at the central reference laboratory using the trained second machine learning model.

22-26. (canceled)
